SEQUENCE LISTING (3 sequences)

SEQ ID NO: 1 - DNA, 20 bp, Lactobacillus plantarum
agagtttgatcatggctcag

SEQ ID NO: 2 - DNA, 22 bp, Lactobacillus plantarum
tagggttaccttgttacgactt

SEQ ID NO: 3 - DNA, 1371 bp, Lactobacillus Plantarum
tgcttgcatcatgatttacatttgagtgagtggcgaactggtgagtaacacgtgggaaacctgcccagaagcgggggataacacctggaaacagatgctaataccgcataacaacttggaccgcatggtccgagcttgaaagatggcttcggctatcacttttggatggtcccgcggcgtattagctagatggtggggtaacggctcaccatggcaatgatacgtagccgacctgagagggtaatcggccacattgggactgagacacggcccaaactcctacgggaggcagcagtagggaatcttccacaatggacgaaagtctgatggagcaacgccgcgtgagtgaagaagggtttcggctcgtaaaactctgttgttaaagaagaacatatctgagagtaactgttcaggtattgacggtatttaaccagaaagccacggctaactacgtgccagcagccgcggtaatacgtaggtggcaagcgttgtccggatttattgggcgtaaagcgagcgcaggcggttttttaagtctgatgtgaaagccttcggctcaaccgaagaagtgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagtatgggtagcaaacaggattagataccctggtagtccataccgtaaacgatgaatgctaagtgttggagggtttccgcccttcagtgctgcagctaacgcattaagcattccgcctggggagtacggccgcaaggctgaaactcaaaggaattgacgggggcccgcacaagcggtggagcatgtggtttaattcgaagctacgcgaagaaccttaccagtcttgacatactatgcaaatctaagagattagacgttcccttcggggacatggatacaggtggtgcatggttgtcgtcagctcgtgtcgtgagatgttgggttaagtcccgcaacgagcgcaacccttattatcagttgccagcattaagttgggcactctggtgagactgccggtgacaaaccggaggaaggtggggatgacgtcaaatcatcatgccccttatgacctgggctacacacgtgctacaatggatggtacaacgagttgcgaactcgcgagagtaagctaatctcttaaagccattctcagttcggattgtaggctgcaactcgcctacatgaagtcggaatcgctagtaatcgcggatcagcatgccgcggtgaatacgttcccgggccttgtacacaccgcccgtcacaccatgagagtttgtaacacccaaa

(19) United States
(12) Patent Application Publication
(10) Pub. No.: US 2022/0372433 A1
(43) Pub. Date: Nov. 24, 2022

(54) LACTOBACILLUS PLANTARUM HG-23 AND APPLICATION THEREOF

(71) Applicant: SHENZHEN BGI AGRICULTURE APPLICATION INSTITUTE, Shenzhen (CN)

(72) Inventors: Xiaoshuang Dai, Shenzhen (CN); Mingming Wang, Shenzhen (CN); Junli Ren, Shenzhen (CN)

(21) Appl. No.: 17/477,720

(22) Filed: Sep. 17, 2021

(30) Foreign Application Priority Data
May 21, 2021 (CN) 202110555784.4

(51) Int. Cl. C12N 1/20; A61K 35/747; A23L 33/135; A23L 33/00

(52) U.S. Cl. CPC C12N 1/205; A61K 35/747; A23L 33/135; A23L 33/40; C12R 2001/25

(57) ABSTRACT

The disclosure relates to a Lactobacillus plantarum HG-23 and application thereof. The Lactobacillus plantarum HG-23 has been deposited with the China Center for Type Culture Collection as assigned Accession Number CCTCC No.: M2021330. The Lactobacillus plantarum HG-23 of the disclosure can be used for preparing hypolipidemic food or hypolipidemic drugs. The Lactobacillus plantarum HG-23 screened in the disclosure is good in safety, can tolerate gastric acid and bile salts, and survives and colonizes in intestinal tract to regulate and improve the intestinal microecological balance of a host, so as to play a beneficial effect and generate an exact healthy effect; in-vitro and animal tests show that the Lactobacillus plantarum HG-23 has a strong function of reducing triglyceride and cholesterol in blood.

### CROSS REFERENCE TO RELATED APPLICATION

This application takes the benefit of and claims priority of Chinese Patent Application No. 202110555784.4 filed on May 21, 2021, the contents of which are herein incorporated by reference.

### REFERENCE TO A SEQUENCE LISTING

This application contains a Sequence Listing in computer readable form. The computer readable form is incorporated herein by reference.

### TECHNICAL FIELD

The disclosure belongs to the technical field of microbiology, and particularly relates to a Lactobacillus plantarum HG-23 and application thereof in regulating blood lipids.

### BACKGROUND

Hypercholesterolemia, also known as high and low density lipoprotein cholesterol, is a primary risk factor of atherosclerotic cardiovascular diseases. The latest research shows that the main vascular events can be reduced by 21% for every 1 mmol/L reduction in low density lipoproteins of people at the risk of cardiovascular diseases. According to statistics in 2013, there are about 200 million people with critical hypercholesterolemia and 88 million patients with hypercholesterolemia in China.

At present, drug therapy and dietary intervention are mainly used to control and reduce the level of blood lipid. Statin and fibrate drugs are widely used in clinic, and the curative effect of these drugs when in initial use is relatively significant, however, side effects can be generated to different extents with long-term administration to aggravate the health problems of patients, for example, long-term administration of statin drugs easily induces myopathy, rhabdomyolysis, abnormal blood sugar, hepatitis, renal dysfunction and the like, and the long-term administration of fibrate drugs can lead to serious liver injury and increased risk of gallstones. The diet of low-fat or low saturated fat food can effectively reduce the content of cholesterol in serum, but the taste and flavor of low-fat food are relatively simple. This dietary suggestion has still not been accepted by the public or preserved for a long time, and it is easy to rebound without paying attention. Therefore, eating hypolipidemic functional food is a more ideal way to control blood lipid for a long time.

At present, researches on probiotics capable of reducing blood lipid have lasted for nearly half a century. Two strains that are deeply researched for a blood lipid reducing function in China belong to Lactobacillus plantarum, respectively including Lactobacillus plantarum P8 and Lactobacillus plantarum ST-III. 50. L. plantarum P8 comes from a team led by Zhang Heping of Inner Mongolia Agricultural University. Animal tests show that L. plantarum P8 has a significant hypolipidemic effect. The Lactobacillus plantarum ST-III jointly developed by the team led by Benheng Guo who was used to be the vice president of the food College of Northeast Agricultural University and now is general manager of Guangming Dairy Co., Ltd. and Jiangnan University and Jiangnan University has successfully proved that its cholesterol reducing effect through animal experiments and population clinical trials, and has been industrialized to become a probiotic product with independent intellectual property rights of Guangming company.

Therefore, developing safe, effective and blood lipid-regulating Lactobacillus has a wide application prospect in the field of fermented food and medicines.

In view of this, this patent is applied.

### SUMMARY

In order to solve the problems existing in the prior art, the disclosure provides a Lactobacillus plantarum HG-23 and application thereof. The Lactobacillus plantarum HG-23 can tolerate gastric acid and bile salts, survives and colonizes in the intestinal tract to regulate and improve the intestinal microecological balance of a host, regulates blood lipids, and specifically reduces cholesterol and triglycerides.

The objective of the disclosure is to provide a Lactobacillus plantarum HG-23.

Another objective of the disclosure is to provide application of the above Lactobacillus plantarum HG-23.

In the Lactobacillus plantarum HG-23 according to the specific embodiment of the disclosure, the Lactobacillus plantarum HG-23 has assigned Accession Number CCTCC No.: M2021330.

In the application of the Lactobacillus plantarum HG-23 according to the specific embodiment of the disclosure in preparation of food or drugs, the food or drugs are blood lipid lowering food or drugs.

Preferably, the food or drugs are food or drugs for reducing cholesterol or lowering triglyceride.

In the application of the Lactobacillus plantarum HG-23 according to the specific embodiment of the disclosure in preparation of drugs, further, the drug comprises the Lactobacillus plantarum HG-23 and a non-toxic carrier for medicine.

In the application of the Lactobacillus plantarum HG-23 according to the specific embodiment of the disclosure in preparation of drugs, further, the dosage of the drug is selected from at least one of tablets, granules, powders, capsules, suspensions, emulsions and lyophilized preparations; preferably, the drug is a compound probiotic tablet.

The drug of the disclosure also comprises pharmaceutically acceptable auxiliary substances. The pharmaceutically acceptable auxiliary substances include but are not limited to pharmaceutically acceptable carriers, diluents, fillers, binders and other excipients. The carrier includes but is not limited to starch or its derivatives, lactose and polyethylene glycol; the diluent includes but is not limited to starch or its derivatives, lactose, sucrose, vegetable oil, wax and fatty acid; the filler includes but is not limited to talc powder the auxiliary substances can also include preservatives, lubricants, dispersants, flavoring agents, moisturizers, antioxidants, colorants, stabilizers, buffers and pH regulators. The above substances can help the stability of the formula or assist in improving the activity or bioavailability as required, or can produce an acceptable taste or smell in the case of oral administration.

In the application of the Lactobacillus plantarum HG-23 according to the specific embodiment of the disclosure in preparation of food, further, the food comprises the Lactobacillus plantarum HG-23 and food-acceptable additives.

The term “food” used in the disclosure is understood in a broad sense, which can be any forms that can be used, that is to say, except conventional food forms, the food of the disclosure can also be health products, drinks or fermented food, etc.

Preferably, the food is probiotic solid powder. A formula of a probiotic solid powder will be described in detail in examples.

In the application of the Lactobacillus plantarum HG-23 according to the specific embodiment of the disclosure in preparation of food, more further, the probiotic solid powder is prepared from xylooligosaccharides, inulin, fructooligosaccharides, lemon fruit powder and Lactobacillus plantarum HG-23.

In the application of the Lactobacillus plantarum HG-23 according to the specific embodiment of the disclosure in preparation of food or drugs, more further, the Lactobacillus plantarum HG-23 is lyophilized powder; the lyophilized powder is prepared by freezing and vacuum drying the bacterial suspension of the Lactobacillus plantarum HG-23 under the sterile environment with a lyophilizing machine.

Compared with the prior art, the disclosure has the beneficial effects:

(1) The Lactobacillus plantarum HG-23 screened in the disclosure can tolerate gastric acid and bile salts, survives and colonizes in the intestinal tract to regulate and improve the intestinal microecological balance of the host, so as to play its beneficial role and produce exact health effects; specifically, the Lactobacillus plantarum HG-23 can reduce triglycerides and cholesterol in the blood.

(2) The Lactobacillus plantarum HG-23 screened in the disclosure can be used for preparing food and drugs for reducing cholesterol and triglycerides, and the tests show that the obtained food also has the function of reducing cholesterol and triglycerides.

### BRIEF DESCRIPTION OF THE DRAWINGS

In order to more clearly explain the technical solution in the embodiment of the disclosure or in the prior art, the accompanying drawings needed to be used in the embodiment or the description of the prior art will be briefly described below. Obviously, the accompanying drawings in the following description are only some embodiments of the utility model. Other drawings can be made by persons of ordinary skill in the art without creative efforts according to these accompanying drawings.

FIG. 1 shows the degradation rate of Lactobacillus plantarum HG-23 according to example 2 of the disclosure on cholesterol, wherein the abscissa is the group (blank control, commercial strain, HG-23), and the ordinate is the degradation rate of cholesterol in vitro;

FIG. 2 shows a gastric acid resistance of Lactobacillus plantarum HG-23 according to example 4 of the disclosure, wherein the abscissa is time and the ordinate is the survival bacterial count of strain HG-23 in pH 2 simulated gastric fluid;

FIG. 3 shows a bile salt tolerance of Lactobacillus plantarum HG-23 according to example 4 of the disclosure, wherein the abscissa is the time and the ordinate is the OD value of strain HG-23 in MRS culture medium and MRS culture medium containing 0.3% bile salt;

FIG. 4 shows the ability of Lactobacillus plantarum HG-23 to reduce in-vivo TG content according to example 5 of the disclosure, wherein the abscissa represents the group and the ordinate represents the concentration of triglycerides;

FIG. 5 shows the ability of Lactobacillus plantarum HG-23 to reduce in-vivo TC content according to example 5 of the disclosure, wherein the abscissa represents the group and the ordinate representing the concentration of cholesterol;

FIG. 6 shows the results of an animal experiment for degrading triglycerides (TG) by probiotic solid powder according to example 6 of the disclosure, wherein the abscissa represents the group and the ordinate represents the concentration of triglycerides;

FIG. 7 shows the results of an animal experiment for degrading cholesterol (TC) by probiotic solid powder according to example 6 of the disclosure, wherein the abscissa represents the group and the ordinate represents the concentration of cholesterol.

### DESCRIPTION OF THE EMBODIMENTS

In order to make the object, technical scheme and advantages of the disclosure more clear, the technical solution of the disclosure will be described in detail below. Obviously, the described embodiments are only parts of the embodiments of the disclosure but not all the embodiments. Based on the embodiments of the disclosure, all other embodiments obtained by those skilled in the art without creative efforts belong to the protective scope of the disclosure.

The Lactobacillus plantarum HG-23 of the disclosure is a Lactobacillus, which can effectively reduce cholesterols and triglycerides, and can be used as a component of food and drugs to improve the symptoms of human hyperlipidemia.

The new strain Lactobacillus plantarum HG-23 (classification name: Lactobacillus plantarum; latin name: Lactobacillus plantarum) has been deposited in China Center for Type Culture Collection on Apr. 6, 2021. The address of the China Center for Type Culture Collection is Wuhan University, Wuhan, China. It is deposited in China Center for Type Culture Collection with the assigned Accession Number CCTCC No: M2021330; the viability was tested by the China Center for Type Culture Collection on Apr. 6, 2021, and the result was: survival.

In some specific embodiments, in the application of Lactobacillus plantarum HG-23 in the preparation of food or drugs, the food or drugs are cholesterol-reducing food or cholesterol-reducing drugs.

Further, the drug comprises the Lactobacillus plantarum HG-23 and a non-toxic carrier for medicines.

Preferably, the drug is a compound probiotic tablet.

The food comprises the Lactobacillus plantarum HG-23 and food-acceptable additives.

Preferably, the food is a probiotic solid powder.

Further, the probiotic solid powder is prepared from xylooligosaccharide, inulin, fructooligosaccharide, lemon fruit powder and Lactobacillus plantarum HG-23; the Lactobacillus plantarum HG-23 is a lyophilized powder; the lyophilized powder is prepared by freezing and vacuum drying the bacterial suspension of Lactobacillus plantarum HG-23 in a sterile environment with a lyophilizing machine.

Next, the technical solution of the disclosure will be further described in detail through examples and in combination with accompanying drawings. However, the selected examples are only used to illustrate the disclosure but not limiting the scope of the disclosure.

### Example 1 Isolation and Identification of Lactobacillus plantarum HG-23

1. Optimization of Culture Medium Formula

The optimization experiment of culture mediums was carried out in groups, and the best formula of lactobacillus culture medium was obtained:

Formula of 1 L MRS solid culture medium: casein peptone 10.0 g/l, beef extract 10.0 g/l, yeast extract 5.0 g/l, glucose 20.0 g/l, dipotassium hydrogen phosphate 2.0 g/l, Tween 801.0 g/l, triammonium citrate 2.0 g/l, sodium acetate 5.0 g/l, magnesium sulfate 0.1 g/l, manganese sulfate 0.05 g/l, agar 17.5 g; sterilize for 20 min at 121° C. under pH 6.5.

Formula of MRS broth culture medium: casein peptone 10.0 g/l, beef extract 10.0 g/l, yeast extract 5.0 g/l, glucose 20.0 g/l, dipotassium hydrogen phosphate 2.0 g/l, Tween 801.0 g/l, triammonium citrate 2.0 g/l, sodium acetate 5.0 g/l, magnesium sulfate 0.1 g/l, manganese sulfate 0.05 g/1; sterilize for 20 min at 121° C. under pH 6.5.

2. Screening of Strains

The strains of Lactobacillus plantarum HG-23 were derived from healthy human intestinal tract, and isolated from the excrements of healthy adults. The excrement samples of healthy adults were diluted by 10′ times in 10-fold gradient with sterile normal saline, coated to the MRS solid culture medium, cultured in 37° C. incubator for 24-48 h, the morphological characteristics of colonies were observed, pick out the suspected colonies of Lactobacillus on the MRS solid culture medium were picked and then transferred respectively to the MRS liquid culture medium for pure culture, and a total of 5 strains were isolated, each of strains was numbered.

The acid and bile salt tolerance and cholesterol reducing ability of several isolated and purified strains were determined. Detailed experiment steps are seen in example 2 and example 3. Lactobacillus plantarum HG-23 having strong acid and bile salt tolerance and cholesterol lowering ability was screened.

3. Identification of Strains

The total bacterium DNA was extracted from Lactobacillus plantarum HG-23 and then subjected to 16s rDNA amplification. The primers were as follows:

sgF:5′-AGAGTTTGATCATGGCTCAG-3′ (SEQ ID NO: 1)

sgR:5′-TAGGGTTACCTTGTTACGACTT-3′ (SEQ ID NO: 2)

These primers were used for PCR amplification and agarose gel electrophoresis, and then glued, recovered and sequenced.

The 16s DNA sequencing results of the isolated strains are shown in SEQ ID No: 3.

### Example 2 In-Vitro Cholesterol Removal Rate Test of Lactobacillus plantarum HG-23

The in-vitro cholesterol removal rate of the strains was detected according to the following steps, and the commercial strain Lactobacillus plantarum 299V was used as experimental control group, especially as follows:

The tested strains were transferred to MRS liquid culture medium, cultured at 37° C. for 24 h, passaged for twice with 1% inoculation amount, and cultured at 37° C. for 15 h for later use. The liquid MRS culture medium was prepared and sterilized for later use.

The MRS culture medium containing cholesterol (culture medium MRSO-CHOL) was prepared: the culture medium is composed of MRS liquid culture medium, sodium mercaptoacetate, bile salt and cholesterol, wherein the concentration of sodium mercaptoacetate in the culture medium MRSO-CHOL was 2 g/1, the concentration of the bile salt in the culture medium MRSO-CHOL was 0.3%, and the concentration of cholesterol was 120 ug/ml.

Experimental group: the Lactobacillus plantarum HG-23 bacterial solution was activated and then incubated into 10 ml of culture medium MRSO-CHOL based on 1% inoculation amount.

Positive control group: the bacterial solution of commercial strains was activated and then inoculated in 10 ml of culture medium MRSO-CHOL based on 1% inoculation amount.

Blank control group: 10 ml of culture solution MRSO-CHOL without strains.

After the two groups of samples were cultured in a 37° C. constant temperature incubator for 24 h, the samples were taken out and uniformly shaken, centrifuged at 4000 r/m for 15 min, the supernatant was taken, the content of cholesterol in the supernatant was determined with an o-phthalaldehyde color developing agent, and the cholesterol removal rate was calculated.

Wherein, the cholesterol removal rate=a difference between cholesterol in control group and cholesterol in experimental group/the content of cholesterol in control group.

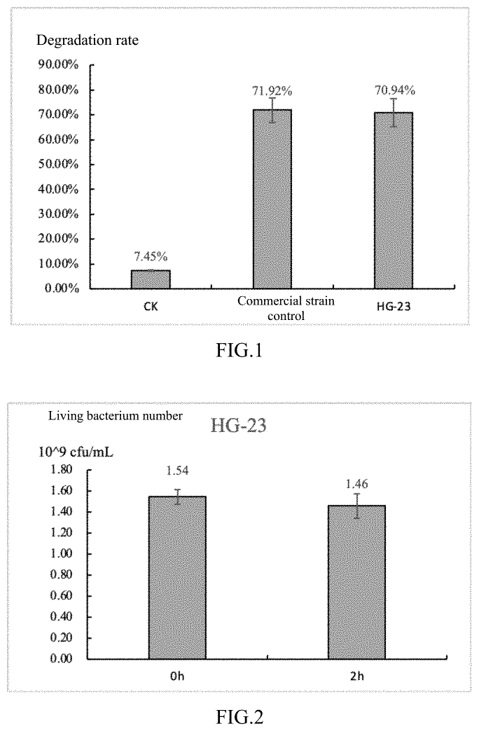

The experimental results are shown in FIG. 1. The cholesterol reducing rate of Lactobacillus plantarum HG-23 is 70.94%, indicating that Lactobacillus plantarum HG-23 has strong in-vitro cholesterol reducing ability.

### Example 3 Bacterial Safety Test of Lactobacillus plantarum HG-23

(1) Antibiotic Sensitivity Test

The antibiotic resistance of HG-23 was detected by a microdilution method. a series of 2-fold dilutions were performed on antibacterial drugs to be tested (Gentamicin, Kanamycin, Tetracycline, Erythromycin, Clindamycin, Chloramphenicol, Amplicilin, with initial concentrations of 1024 ug/ml, 256 ug/ml, 64 ug/ml, 8 ug/ml, 16 ug/ml, 64 ug/ml, 16 ug/ml) for 12 times, then added into corresponding wells of 96-well plates, and then 2 ml diluted test bacterial solutions were added into the corresponding wells. After being cultured for 48 h, the minimum inhibitory concentration (MIC) well of the antibacterial drug, namely the sensitivity of the test strain, was observed. The experimental results are shown in Table 1.

TABLE 1
Resistance test results of Lactobacillus plantarum
HG-23 to antibiotics

| | | EFSA specified safety value |
|---|---|---|
| Antibiotics (mg/mL) | HG-23 | (Lactobacillus plantarum) |
| Gentamicin | 8 | 16 |
| Kanamycin | 32 | 64 |
| Tetracycline | 16 | 32 |
| Erythromycin | 0.5 | 1 |
| Clindamycin | 0.5 | 2 |
| Chloramphenicol | 2 | 8 |
| Amplicilin | 1 | 2 |

It can be seen from table 1 that the resistance test results of Lactobacillus plantarum HG-23 to antibiotics show that the MIC of antibiotics is within the safe range.

(2) Toxicity Test of Metabolites

Lactic acid optical activity test: a D-/L-lactic acid test kit from Ireland Megazyme company was used. According to the experimental results, D-lactic acid is not produced.

Detection of nitrate reductase activity: under sterile conditions, the activated strains were incubated into a nitrate culture medium based on 3% of inoculated amount and cultured for 5d at 37° C., then 10 drops of potassium iodide solution and 10 drops of starch solution were added respectively, and the experimental results were observed. At the same time, the blank experiment was carried out. The experimental results show that all the bacterial solutions do not turn blue, which indicates negative reaction.

Indole experiment: under sterile conditions, the activated strains were incubated into a peptone aqueous medium based on 3% of inoculated amount and cultured for 72 h at 37° C., 8˜10 drops of indole reagent were added, and the experimental results were observed. At the same time, the blank experiment was carried out. The experimental results show that there are no red circles, indicating that its metabolism does not produce indole.

Detection of amino decarboxylase activity: bacteria with amino acid decarboxylase can decompose amino acids so that the amino acids were decarboxylated to produce amines (lysine cadaverine, ornithine putrescine, and arginine arginine) and carbon dioxide, the culture medium become alkaline, and the indicator (bromocresol violet) was dropped. The solution was negative if it was yellow, was positive if it was purple. The results show that the tube was yellow and negative.

(3) Determination of Cell Adhesion Ability

The monolayer culture of cells in a 6-well plate was cultured for 1 h at a constant temperature of 37° C. with incomplete DMEM (without addition of serum and double antibody); the culture solution was sucked, washed with sterile PBS for 3 times, and the washing solution was sucked and dried; 500 μL of 10⁸ cfu/ml experimental strains and 500 μL of incomplete

DMEM were uniformly mixed, and then added into the 6-well plate, and meanwhile blank control with no cells and only bacterial solution was set, and the above substances were cultured for 1.5 h at the constant temperature; the culture plate was taken out, the culture solution was sucked, the culture plate was washed with sterile PBS for 5 times, and the washing solution was sucked up; 10% formaldehyde was added into each well and immobilized for 2 h at room temperature, the immobilization solution was sucked, the culture plate was washed for 3 times with sterile PBS, the washing solution was sucked, the culture plate was dried at room temperature; after gram staining, microscopic examination was performed, 20 visual fields were randomly selected and counted, and the adhesion index was calculated:

Adhesion index=bacteria count/cell number (i.e., the average number of bacteria adhered to each cell).

The 20 visual fields of strains were randomly selected, the total number of bacteria and cells adhered to the cells in the visual field were counted and the adhesion index was measured. The experimental results show that the adhesion index of HG-23 to human colon cancer cell line HT-29 was 5.6, and HG-23 has good cell adhesion ability.

### Example 4 In-Vitro Acid and Bile Salt Tolerance Test of Lactobacillus plantarum HG-23

Simulated gastric fluid SGF (2.0 g NaCl+3.2 g pepsin+an appropriate amount of concentrated hydrochloric acid to adjust the pH value to 3.0) was prepared, and filtered with 0.2 μm microporous membrane for later use. The inclined strains were picked up to be incubated into the MRS liquid culture medium to incubate for 16-18 h at 37° C. The bacteria suspension was centrifuged for 15 min at 4000 r/min, the supernatant was discarded, and the wet weight of the bacteria was weighed. The bacteria were re-suspended in normal saline in the ratio of 0.1 g/ml, the obtained re-suspension was added into the SGF solution in a ratio of 1:10, sufficiently and evenly mixed and then cultured for 2 h in a 37° C. constant temperature incubator, and the cells were counted. The experimental results are shown in FIG. 2. The number of viable bacteria remains at an order of magnitude, indicating that Lactobacillus plantarum HG-23 has strong acid resistance in vitro.

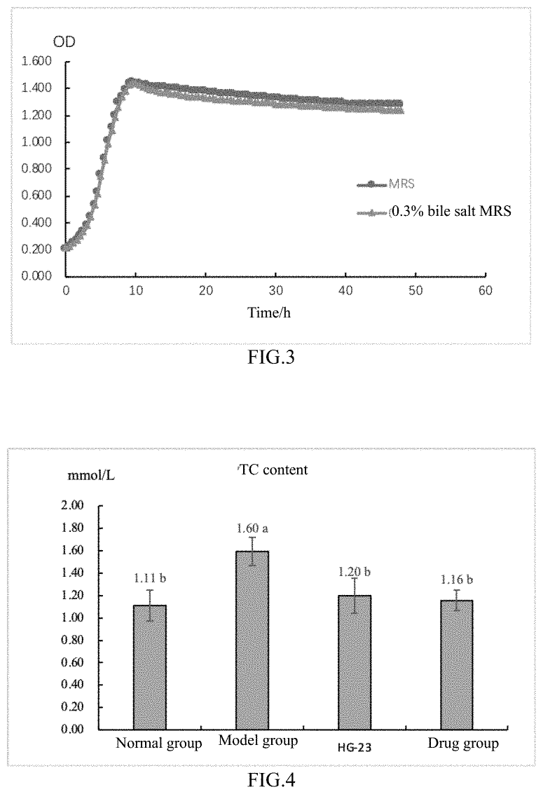

The twice activated bacterial solution was incubated into 10 mL of sterilized MRS liquid culture medium containing 0.3% bile salt to be cultured, and the growth curve was measured. The experimental results are shown in FIG. 3, indicating that HG-23 has strong bile resistance in vitro.

### Example 5 In-Vivo Effectiveness Study of Lactobacillus plantarum HG-23 (Animal Experiment)

### (1) Preparation of Experimental Strains

The twice activated Lactobacillus plantarum HG-23 was inoculated into the MRS liquid culture medium, cultured at 37° C. for 18 h, centrifuged at 6000 r/m for 10 min, washed with sterilized normal saline and collected. Then, 0.85% normal saline was added and the number of bacteria was adjusted to about 1.0×109 CFU/ml, and then the viable bacteria were sub-packaged into 15 ml centrifuge tubes based on daily dosage. Administration dosage was 2 ml/mouse per day, 10 mice in each group, 4 groups in total, and gavage lasted for 28 days.

### (2) Grouping and Feeding Methods of Experimental Animals

Sprague-Dawleys were 5-year-old male rats, 40 rats in total, were fed freely to d28. They were divided into 4 groups with 10 rats in each group:

HG-23 group: viable bacteria suspension was gavaged and high-fat feed was fed;

Model group: 0.85% normal saline was gavaged and high-fat feed was fed;

Normal group: 0.85% normal saline was gavaged and standard feed was fed;

Xuezhikang group: standard dose Xuezhikang was gavaged and high-fat feed was fed.

Each rat was given 2 ml by gavage every day for 28 days, and then stopped gavage. The feed and water of each group remained unchanged for 28 days.

### (3) Sample Collection and Analysis Test

Blood was collected before the formal test, on the d28 and at the time period. The blood collection method was that blood was taken from the femoral vein of rats after fasting for one night, the blood was centrifuged at 4000 r/m for 10 min after coagulation, the serum was separated, and cholesterol and triglyceride were detected respectively by using the kit (name of the kit: total cholesterol determination kit, company: Zhejiang Dongou diagnostic products Co., Ltd.) and chromotropic acid chromogenic method and enzyme labeling instrument (company: molecular devices, model: spectrum max190).

### (4) Experimental Results

The experimental results are shown in FIG. 4 and FIG. 5. After 28 days of feeding high-fat rats with bacterial suspension containing HG-23 strains, the blood was drawn via femoral vein for detection, it was found that compared with control group, the contents of triglyceride (TG) and cholesterol (TC) in the blood of rats were respectively reduced by 25% and 27.6%, indicating that the Lactobacillus plantarum HG-23 has the function of reducing cholesterol and triglyceride in rats.

### Example 6

Preparation of blood lipid-reducing compound probiotic solid powder. The compound probiotic solid powder is composed of the following components by weight percent: 30% of lemon fruit powder, 22% of fructooligosaccharide, 20% of xylooligosaccharide, 20% of inulin and 8% of Lactobacillus plantarum HG-23 powder.

Animal experiments show that Lactobacillus plantarum HG-23 can effectively reduce the levels of cholesterol and triglyceride in serum. Fructooligosaccharides, xylooligosaccharides and inulin are prebiotics, which can promote the proliferation of probiotics. In addition, many studies at home and abroad have shown that fructooligosaccharides, xylooligosaccharides and inulin can reduce the concentrations of triglycerides and cholesterol in serum, and then improve lipid metabolism. The formula also has the advantages of natural components, scientific formula, balanced nutrition, convenient administration, good solubility and non-stick teeth.

The compound probiotic solid powder is prepared according to the following process steps:

(1) preparation of lyophilized powder: the suspension of Lactobacillus plantarum HG-23 was frozen and vacuum dried in a sterile environment with a lyophilizing machine to prepare Lactobacillus plantarum HG-23 lyophilized powder, with a vacuum degree of 6 Pa, a freezing temperature of −40° C., and a lyophilizing time of 7-10 h. The number of viable bacteria per gram of Lactobacillus plantarum HG-23 lyophilized powder was 5×10¹¹ cfu, and the moisture content was less than 5%;

(2) ingredients: Lactobacillus plantarum HG-23, lemon fruit powder, fructooligosaccharide, xylooligosaccharide, inulin and raw materials were transferred to 100000 level clean area, and samples were inspected;

(3) screening: after the inspection was qualified, the samples were screened in 80 mesh respectively, and the raw materials were weighed according to the proportion;

(4) mixing: the weighed raw materials were mixed while stirring to obtain total mixed powder with consistent color and no color difference; and

(5) packaging: the mixed powder was packaged, labeled and packed according to the measured weight to obtain the finished product.

The blood lipid-reducing compound probiotic solid powder obtained in example 6 was subjected to efficacy trial.

Test solution: the test solution is the same as that in example 5, in which the sample for gavage was changed into the suspension made of the above probiotic solid powder and sterile water, and the number of bacteria was adjusted to about 1×10⁹ CFU/ml, the administration dosage was 2 ml/rate per day.

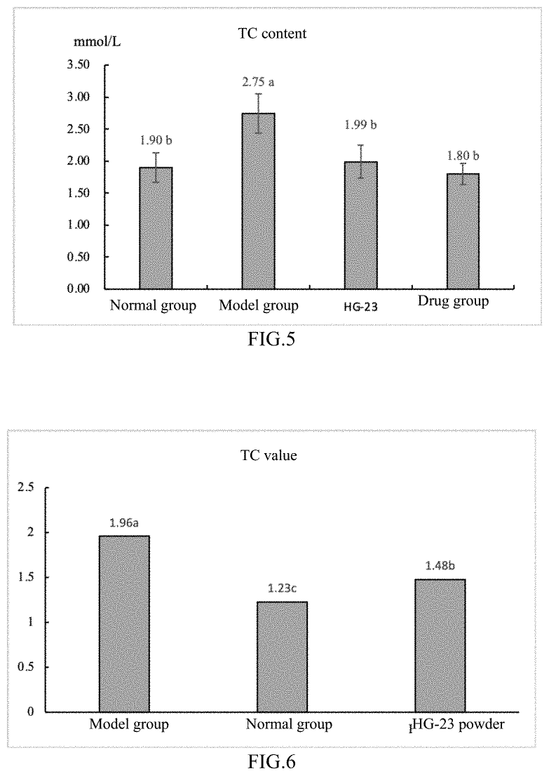

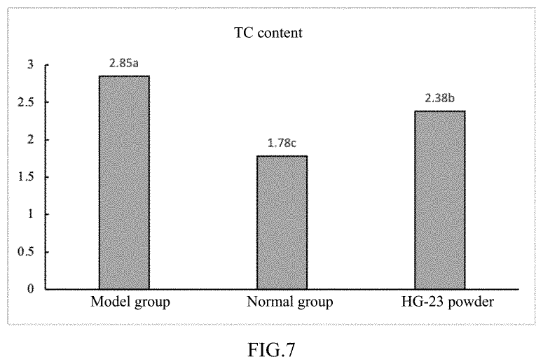

After high-fat rats were fed with the suspension of the above probiotic solid powder for 28 days, blood was drawn from femoral veins and tested. The results are shown in FIG. 6 and FIG. 7, it was found that compared with the control, triglycerides and cholesterol in the blood of rats were decreased significantly, and Lactobacillus plantarum HG-23 was colonized and played a role in the rats, indicating that the above probiotic solid powder has the effect of reducing triglyceride and cholesterol in rats after gavaged.

The above description is only the specific embodiment of the disclosure, but the protective scope of the disclosure is not limited thereto. Any person skilled in the art can easily conceive those changes or replacements within the technical scope disclosed by the disclosure should be included within the protective scope of the disclosure. Therefore, the protective scope of the disclosure shall be subject to the protective scope of the claims.

What is claimed is:

1. A Lactobacillus plantarum HG-23, the Lactobacillus plantarum HG-23 having the assigned Accession Number CCTCC No.: M2021330.

2. Application of the Lactobacillus plantarum HG-23 of claim 1 in preparation of food or drugs.

3. The application according to claim 2, wherein the food or drugs are hypolipidernic food or hypolipidemic drugs.

4. The application according to claim 2, wherein the food or drugs are used for reducing cholesterol or reducing triglyceride.

5. The application according to claim 3, wherein the drug comprises the Lactobacillus plantarum HG-23 of claim 1 and a non-toxic carrier for medicine.

6. The application according to claim 5, wherein the dosage of the drug is selected from at least one of tablets, granules, powders, capsules, suspensions, emulsions and lyophilized preparations;

7. The application according to claim 5, wherein the drug is a compound probiotic tablet.

8. The application according to claim 3, wherein the food comprises the Lactobacillus plantarum HG-23 of claim 1 and food-acceptable additives; preferably, the food is probiotic solid powder.
